(19) United States
(12) Patent Application Publication
(10) Pub. No.: US 2014/0058372 A1
(43) Pub. Date: Feb. 27, 2014

(54) TREATMENT FOR RENAL FAILURE

(71) Applicant: Amir Belson, Los Altos, CA (US)

(72) Inventor: Amir Belson, Los Altos, CA (US)

(21) Appl. No.: 13/973,803

(22) Filed: Aug. 22, 2013

Related U.S. Application Data
(60) Provisional application No. 61/692,163, filed on Aug. 22, 2012.

(51) Int. Cl. A61N 1/36; A61B 18/14; A61N 1/05

(52) U.S. Cl. CPC A61N 1/36007; A61N 1/0551; A61B 18/14
USPC 606/32; 607/118; 607/59

(57) ABSTRACT

A method of increasing renal function in a patient operates by stimulation of perivascular sympathetic nerves found in the vicinity of the hepatic portal vein and the hepatic artery. The method can be used as a treatment for renal failure or chronic kidney disease. Alternatively, the method can be used as a prophylactic treatment for preventing contrast-induced nephropathy or any other toxic nephropathy, which can result in renal failure. The perivascular sympathetic nerves can be stimulated by applying energy, such as electrical energy, light, vibration, and ultrasonic vibration, to the perivascular sympathetic nerves. Various methods are described for stimulating the perivascular sympathetic nerves using electrodes that are placed using minimally-invasive techniques.

### CROSS REFERENCE TO OTHER APPLICATIONS

This application claims the benefit of U.S. Provisional Application No. 61/692,163, filed on Aug. 22, 2012.

### FIELD OF THE INVENTION

The present invention relates generally to treatments for renal failure, which is also known as chronic kidney disease. More specifically, it relates to a method of treatment for renal failure that operates by electrical stimulation of perivascular sympathetic nerves.

### BACKGROUND OF THE INVENTION

Chronic kidney disease (CKD) is a worldwide public health problem. It is recognized as a common condition that is associated with an increased risk of cardiovascular disease and chronic renal failure (CRF). In the United States, there is a rising incidence and prevalence of kidney failure, with poor outcomes and high cost. Kidney disease is the ninth leading cause of death in the United States.

The Third National Health and Examination Survey (NHANES III) estimated that the prevalence of chronic kidney disease in adults in the United States was 11% (19.2 million): 3.3% (5.9 million) had stage 1, 3% (5.3 million) had stage 2, 4.3% (7.6 million) had stage 3, 0.2% (400,000) had stage 4, and 0.2% (300,000) had stage 5.

The prevalence of chronic kidney disease stages 1-4 increased from 10% in 1988-1994 to 13.1% in 1999-2004. This increase is partially explained by the increase in the prevalence of diabetes and hypertension, the two most common causes of chronic kidney disease. Data from the United States Renal Data System (USRDS) indicated that the prevalence of chronic renal failure increased 104% between the years 1990-2001.

According to the Third National Health and Nutrition Examination Survey, it was estimated that 6.2 million people (i.e. 3% of the total US population) older than 12 years had a serum creatinine value above 1.5 mg/dL; 8 million people had a GFR of less than 60 mL/min, the majority of them being in the Medicare senior population (5.9 million people).

The Kidney Disease Outcomes Quality Initiative (K/DOQI) of the National Kidney Foundation (NKF) defines chronic kidney disease as either kidney damage or a decreased glomerular filtration rate (GFR) of less than 60 mL/min/1.73 m² for 3 or more months. Whatever the underlying etiology, the destruction of renal mass with irreversible sclerosis and loss of nephrons leads to a progressive decline in GFR. The different stages of chronic kidney disease form a continuum in time.

In 2002, K/DOQI published its classification of the stages of chronic kidney disease, as follows:
Stage 1: Kidney damage with normal or increased GFR (>90 mL/min/1.73 m²)
Stage 2: Mild reduction in GFR (60-89 mL/min/1.73 m²)
Stage 3: Moderate reduction in GFR (30-59 mL/min/1.73 m²)
Stage 4: Severe reduction in GFR (15-29 mL/min/1.73 m²)
Stage 5: Kidney failure (GFR<15 mL/min/1.73 m² or dialysis)

The goal of current treatments for chronic kidney disease is to slow down or halt the progression of CKD to stage 5. Control of blood pressure and treatment of the underlying disease, whenever feasible, are the broad principles of management. Generally, angiotensin converting enzyme (ACE) inhibitors or angiotensin II receptor blockers (ARB) are used to reduce blood pressure, which has been found to slow the progression of CKD to stage 5. Diuretics are typically also prescribed to increase GFR and to further reduce blood pressure by reducing fluid retention. Other symptoms and concomitant conditions may also need to be treated, including hyperlipidemia, anemia, bone disease and diabetes mellitus. When a patient reaches stage 5 CKD (also known as end-stage renal failure), renal replacement therapy is usually required, in the form of either dialysis or a transplant. While renal replacement therapies can maintain patients indefinitely and prolong life, the quality of life is severely affected. Renal transplantation increases the survival of patients with stage 5 CKD significantly when compared to other therapeutic options; however, it is associated with an increased short-term mortality due to complications of the surgery.

Diuretics, which are part of the primary drug therapy for renal failure, can cause quite a number of potential complications, including electrolyte imbalances, acid-base imbalances, hypokalemia, hyperkalemia, hyponatremia, ototoxicity, arrhythmia, and glucose intolerance. Reported idiosyncratic reactions to diuretics include interstitial nephritis, noncardiogenic pulmonary edema, pancreatitis, and myalgias. Side effects can include fatigue, muscle cramps, dizziness, dehydration, skin rash, nausea, sore throat and fever. Control or avoidance of these complications may require a change in drug regimen and/or frequent adjustments to the dosage of diuretics taken. In addition, some patients are resistant or become resistant to the therapeutic effects of diuretics.

Patient non-compliance is frequently a problem in diuretic therapy. Diuretic therapy causes frequent urination. This should not be considered a side-effect as it is, in fact, the primary mechanism of therapeutic action for these drugs. The need for frequent urination can be disruptive of a patient's sleep patterns and can interfere with many of the patient's daily activities. Consequently, many patients try to reduce the dosage of diuretics taken or avoid them altogether.

Congestive heart failure is another chronic condition for which diuretics are routinely prescribed in order to reduce fluid retention and blood pressure. Congestive heart failure is often associated with chronic kidney disease and particularly with end-stage renal failure.

Contrast-induced nephropathy can cause acute or chronic renal failure or it can exacerbate underlying renal disease. Radiopaque contrast agents, which are used in angiography and fluoroscopy-guided interventional procedures, have a nephrotoxic effect. When used in large doses and/or on patients with compromised renal function, permanent renal damage can occur. Many of the newer, more complicated interventional procedures, such as transcatheter aortic valve replacement (TAVR), frequently require a large volume of contrast agent to be used, particularly at the beginning of the learning curve for new procedures. Anticipation and avoidance of contrast-induced nephropathy is a far better strategy than treating the resulting renal failure after it occurs.

### SUMMARY OF THE INVENTION

The present invention provides a method of increasing renal function in a patient. In a first aspect, the present invention provides a treatment for renal failure or chronic kidney disease. In a second aspect, the invention provides a prophylactic treatment for preventing contrast-induced nephropathy or any other toxic nephropathy, which can result in renal failure. The method of treatment operates by stimulation of perivascular sympathetic nerves, for example by applying energy, such as electrical energy, light, vibration, and ultrasonic vibration, to the perivascular sympathetic nerves.

### DESCRIPTION OF THE INVENTION

The present invention provides a method of increasing renal function in a patient. The method can be used as a treatment for renal failure or chronic kidney disease. Alternatively, the method can be used as a prophylactic treatment for preventing contrast-induced nephropathy or any other toxic nephropathy, which can result in renal failure. The method of treatment operates by stimulation of perivascular sympathetic nerves, for example by electrical stimulation of the perivascular sympathetic nerves using electrodes that are placed using minimally-invasive techniques.

It has been shown that electrical stimulation of certain perivascular sympathetic nerves, in particular those surrounding the hepatic portal vein and the hepatic artery, can have an effect on renal function (Pfliigers Arch (1993) 425:268-271, Eur. J. Biochem. 158,13-18 (1986)). During electrical stimulation, renal output decreased, as shown by a drop in glomerular filtration rate (GFR) of about 45% from baseline. In the period after electrical stimulation, renal output increased, as shown by a rise in glomerular filtration rate (GFR) of about 20% from baseline. This effect lasted for approximately 10 minutes after electrical stimulation.

To take advantage of this physiological effect, the method of the present invention places an electrode catheter or leadless electrodes in the lumen of the hepatic portal vein and/or the hepatic artery. Preferably, the electrodes are configured so that they do not impede the flow of venous or arterial blood through the lumens of the vessels. The electrodes are preferably placed in the lumens of the vessels using minimally-invasive techniques. Alternatively, the electrodes can be placed external to the lumens of the vessels in the vicinity of the portal vein and/or the hepatic artery, preferably using minimally-invasive techniques. Various minimally-invasive approaches can be used:

Percutaneous transhepatic approach—The portal vein is accessed using an elongated needle to puncture the skin and the abdominal wall overlying the liver and to subsequently puncture the liver itself until a branch of the intrahepatic portal venous system is accessed. This puncture and catheter placement is preferably achieved with the guidance of an imaging modality, such as ultrasound (e.g. two-dimensional or Doppler flow imaging), radiographic imaging (e.g. fluoroscopy or computed tomography), or magnetic resonance imaging. Conventional percutaneous access techniques such as guidewire manipulations and introducer sheath insertions may then be utilized for the placement of the catheter into the portal venous system for movement into the appropriate position for delivery of an electrode catheter or leadless electrodes into the portal vein.

Intravenous Intrahepatic Approach—The portal vein is accessed using a catheter that is introduced into a peripheral vein, such as the jugular or femoral vein. The catheter is advanced into the intrahepatic venous system, preferably using guidewire techniques and under the guidance of ultrasound imaging (e.g. two-dimensional or Doppler flow imaging), radiographic imaging (e.g. fluoroscopy or computed tomography), or magnetic resonance imaging. A puncture through the intrahepatic venous structure, through the liver parenchyma and into an intrahepatic portal venous structure, is then accomplished. Catheter exchanges using conventional techniques may then be performed for the placement of an electrode catheter or leadless electrodes into the portal vein using this intrahepatic access technique.

Arterial catheterization—The hepatic artery can be accessed using standard arterial catheterization techniques using fluoroscopic guidance. A catheter is introduced into a peripheral artery, such as the femoral artery or brachial artery, and advanced into the abdominal aorta. The tip of the catheter is directed into the celiac trunk and then into the common hepatic artery and the hepatic artery proper using a guidewire and/or a curved or steerable catheter tip. An electrode catheter or leadless electrodes can be placed into the hepatic artery through the catheter or using a catheter exchange.

Endoscopic Approach—Both the hepatic artery and the portal vein run parallel to one another in close proximity to the stomach. A flexible endoscope or gastroscope is introduced through the esophagus into the patient's stomach. An elongated needle inserted through a working channel in the endoscope is used to puncture the stomach wall and, optionally, the wall of the hepatic artery or the portal vein. An electrode catheter or leadless electrodes can be placed into the hepatic artery or the portal vein or, alternatively, in the perivascular area close to the hepatic artery and/or the portal vein.

Laparoscopic Approach—The hepatic artery, the portal vein or the perivascular area can be directly accessed through a laparoscopic puncture in the abdominal wall. Optionally, the peritoneum can be insufflated to create more working space within the abdomen. An electrode catheter or leadless electrodes can be placed into the hepatic artery or the portal vein or in the perivascular area close to the hepatic artery and/or the portal vein.

In one embodiment of the invention, an electrode catheter with one or more electrodes can be inserted into or in the vicinity of the hepatic artery or the hepatic portal vein. Preferably, the electrodes are configured so that they do not impede the flow of venous or arterial blood through the lumens of the vessels. For example, the electrodes may be configured to be expandable like a wire mesh stent. The stent-like electrodes can be selectively expanded to contact the vessel walls, but the open space inside of the expanded electrodes will allow the blood to flow unimpeded through the vessel. The stent-like electrodes can be self-expanding, mechanically expandable or balloon expandable. Other possible electrode configurations include a conical, spiral, helical or cylindrical array of struts, legs wires or needles. Alternatively, a needle puncture can be made in the vessel wall and the electrode implanted into the perivascular area adjacent to the perivascular nerves. The electrode catheter has a conductive wire that connects each of the electrodes to a proximal connector that is configured to make an electrical connection with an electronic module. The electronic module contains a battery or other energy source and circuitry for delivering a course of electrical stimulation to the electrodes. The electronic module is preferably small enough that it can be implanted subcutaneously or intra-abdominally without discomfort or inconvenience to the patient. The electrode catheter and the electronic module can be temporarily or permanently implanted. The electrodes and electrical leads can be anchored or not anchored to surrounding tissue or structures.

In an alternative embodiment that is intended for temporary use, one or more electrodes can be mounted on one or more inflatable balloons or other selectively expandable structure. During the stimulation period, the balloons can be inflated to press the electrodes into contact with the vessel wall. In between stimulation periods, the balloons can be deflated.

In another embodiment of the invention, one or more wireless or leadless electrodes can be inserted into or in the vicinity of the hepatic artery or the portal vein. Preferably, the electrodes are configured so that they do not impede the flow of venous or arterial blood through the lumens of the vessels. In one example, the electrodes may be expandable stent-like structures, as described above. Alternatively, the electrodes may simply be small enough in diameter that they can be place against or inserted into the vessel wall without impeding the flow of blood through the vessel.

The leadless electrodes can be configured in various ways. In one embodiment, the leadless electrodes are configured as part of a unitary, self-contained electrical stimulation device. The electrical stimulation device has a housing that contains a battery or other power source and circuitry for delivering a course of electrical stimulation to the electrodes. Optionally, the electrical stimulation device may be configured for wireless communication with an external device for recording data and controlling and/or programming the electrical stimulation device. In another embodiment, the leadless electrodes may be configured to receive power for electrical stimulation from an external source, such as an implantable or wearable electronic module. Energy is delivered wirelessly from the electronic module to the electrodes, for example by electrical induction, ultrasonic energy or optical energy. The leadless electrodes will have components and circuitry to receive the energy and convert it to electrical impulses for stimulating the nerves. The electrodes and the electronic module can be temporarily or permanently implanted. Alternatively, the electronic module can be external to the patient and the energy delivered transcutaneously from the electronic module to the electrodes.

Alternatively, ultrasonic energy, magnetic energy or optical energy (e g infrared light or laser energy) may be used to directly stimulate the perivascular nerves. Optical or light energy can be delivered to the target nerves from a remote or external source using an optical fiber or, alternatively, a light source such as a light emitting diode or diode laser can be positioned in the vicinity of the perivascular nerves.

The leadless electrode can be delivered as mentioned to the desired location using an endoscope that will be inserted through the esophagus and stomach and then a catheter will be introduced through the working channel of the endoscope, preferably under guidance using ultrasound or another imaging modality. In one embodiment the endoscope will have an ultrasound probe for imaging on its distal end. The catheter will be guided through the wall of the stomach or the duodenum to the desired location. The catheter will deliver the leadless electrode to the desired location.

The leadless electrodes can be configured as part of a unitary, self-contained electrical stimulation device, as described above, or, alternatively, a separate electronic module or pulsing unit that will wirelessly activate the leadless electrode(s) can be implanted under the skin in another desired location.

The electronic module can be made programmable. For example, a regimen of periodic electrical stimulation of the perivascular nerves can be administered during the day to increase GFR and the electrical stimulation can be reduced or stopped at night so as not to interfere with the patient's sleep patterns. The electronic module can be configured so that increases, decreases or other changes to the electrical stimulation regimen can be programmed transcutaneously. Optionally, the electronic module may be configured for wireless communication with an external device for recording data and controlling and/or programming the electrical stimulation functions.

For treatment of chronic renal failure, it is preferable that the electrodes or electrode catheter and the electronic module be permanently implanted. For prophylactically treating contrast-induced nephropathy, it is preferable that the electrodes or electrode catheter and the electronic module be placed temporarily. For example, an electrode catheter can be placed into the hepatic artery using a minimally invasive catheter-based technique prior to an interventional procedure that is expected to use a large volume of radiopaque contrast agent, particularly in patients known to have renal impairment or other risk factors. Periodic electrical stimulation of the perivascular nerves can be begun before the procedure to increase GFR and continued after the procedure for as long as necessary to clear the contrast agent from the patient's circulatory system. Hydration therapy can be used in conjunction with electrical stimulation.

One method that has been found to be effective utilizes a bipolar electrode configuration for delivering 2 millisecond pulses of 20 Volts at 20 Hz delivered over a 1 to 2 minute period with a 10 minute interval between stimulation periods. A monopolar electrode configuration may also be used, with the electronic module having a ground electrode or a grounded outer housing for completing the electrical circuit. A catheter with multiple electrodes can be used to test what electrode configuration and positioning will provide the optimal results.

Since GFR actually increases during the interval between stimulation periods, the duration and the ratio of electrical stimulation to the interval between stimulation periods can be adjusted to optimize the therapeutic effect. The interval between stimulation periods will preferably be at least 2.25 times longer than the period of nerve stimulation, more preferable from 5 to 10 times longer or more, in order to achieve an increase in renal output.

Various regimens of energy can be used to stimulate the perivascular nerves. The stimulation energy can be applied continuously or intermittently during the stimulation period. The stimulation energy can be applied at a steady energy level or a variable level, for example in the form of various wave shapes, including sine waves, square waves or other more complex waveforms. Stochastic (i.e. random) application of energy during the stimulation periods may also be utilized. The stimulation energy can be applied differently at different time points, e.g. continuous for an initial period followed by intermittent at later time points. The application of stimulation energy can be triggered based on elapsed time, absolute time, time of day, external stimuli or a response to a sensor that senses either electrical activity or certain chemical species.

In an alternate method, an electrode catheter or the like can be utilized for permanently disabling or ablating the perivascular sympathetic nerves, in particular the nerves surrounding the hepatic portal vein and/or the hepatic artery, to achieve a permanent increase in renal function. Electrical stimulation of the perivascular sympathetic nerves can be used to find the optimal electrode locations to achieve the desired therapeutic effect, then higher energy pulses can be delivered through the electrodes to permanently disable or ablate the perivascular sympathetic nerves. The higher energy pulses can be radiofrequency energy or, alternatively, ultrasonic energy or optical energy (e.g. laser energy) may be used. In this method, permanent implantation of the electrodes and electronic module may be unnecessary. Chemical or pharmaceutical ablation of the perivascular sympathetic nerves may also be used.

Alternatively, a cryogenic catheter probe can be used to temporarily disable the perivascular sympathetic nerves to verify an increase in renal function and identify the best location for ablating the perivascular sympathetic nerves. Cryogenic ablation or high energy ablation techniques, as described above, can be utilized for permanently disabling or ablating the perivascular sympathetic nerves to achieve a permanent increase in renal function.

A stimulation device for carrying out the method of the present invention can take one of several different forms. The stimulation device can be configured for temporary use, which would be applicable for preventing contrast induced nephropathy, or it can be configured to be implantable, which would be more applicable for treating chronic renal failure.

A stimulation device for temporary use would generally have a first configuration to facilitate insertion into a blood vessel, such as a hepatic portal vein or hepatic artery, or through a catheter placed in a blood vessel and would be selectively or automatically expandable toward a second configuration where one or more electrodes would engage a side wall of the blood vessel. One or more conductive wires would connect the electrode(s) to an electronic module. The electronic module would include an energy source and circuitry for delivering a course of stimulation to the electrode(s). The energy source may be a battery and/or one or more capacitors or, alternatively, an external electrical source may be used.

A stimulation device for long-term implantation would generally have one or more electrodes, an anchoring mechanism for holding the electrode(s) in contact with the target tissue and an electronic module containing an energy source and circuitry for delivering a course of stimulation to the electrode. The anchoring mechanism may include, but is not limited to, one or more barbs, hooks or expanding structures. The energy source will generally include a battery and/or one or more capacitors. Alternatively or in addition, transcutaneous energy transmission can be used to power the electronic module and/or recharge the internal energy source. The electronic module may be integrated with the electrode(s) in a leadless configuration or there may be one or more conductive wires connecting the electrode(s) to the electronic module. The stimulation device may be configured for deployment within or through a blood vessel, such as a hepatic portal vein or hepatic artery, or it may be configured for deployment external to a blood vessel in the vicinity of the perivascular nerves.

Whether for temporary use or implantation, the stimulation device can be monopolar, with a single stimulation electrode and a ground electrode located elsewhere on the device, or bipolar, with two stimulation electrodes. Alternatively, multiple selectively-addressable stimulation electrodes can be used to locate the electrode positions that achieve the best therapeutic effect. The electrode(s) may be selectively or automatically expandable to engage a side wall of the blood vessel or the device may include an expandable mechanism to press the electrode(s) against or even through the vessel wall. The electrode(s) may be constructed in many different configurations including, but not limited to, a flat, annular, spiral, helical, mesh, or stent-like structure. One or more electrodes can be arranged in a conical, cylindrical, spiral or helical array. The electrode(s) may be constructed from materials including, but not limited to, Nitinol, stainless steel, Elgiloy, titanium, MP35N, platinum alloys, gold alloys or combinations thereof.

Optionally, the electrode(s) may be configured to penetrate the vessel wall for more direct electrical contact with the perivascular nerves. For example, one or more penetrating needles may emerge from a hollow portion of the stimulation device to penetrate the vessel wall. The deployment of the penetrating needles from the hollow portion of the stimulation device may be selectively or automatically triggered. The needle(s) may be straight or curved. The needle(s) themselves may serve as electrodes or one or more separate electrodes may be deployed or expanded from within the needle(s).

The stimulation device may include one or more radiopaque and/or echogenic markers to facilitate fluoroscopic and/or ultrasound imaging. The stimulation device may include one or more sensors in proximity to the electrode to monitor nerve activity.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

1. A method of increasing renal function in a patient, comprising:
periodically stimulating a sympathetic nerve located in a perivascular area in the vicinity of a portal vein or hepatic artery of the patient during which glomerular filtration rate of the patient is decreased, each period of nerve stimulation being followed by an interval period with no nerve stimulation during which glomerular filtration rate of the patient is increased, wherein a cumulative increase in glomerular filtration rate during the interval period with no nerve stimulation is greater than a cumulative decrease in glomerular filtration rate during the preceding period of nerve stimulation.

2. The method of claim 1, wherein the nerve stimulation is applied in a daily pattern to increase the glomerular filtration rate during the patient's planned waking hours and not to increase the glomerular filtration rate during the patient's planned sleeping hours.

3. The method of claim 1, wherein the interval period with no nerve stimulation has a duration of at least approximately 2.25 times a duration of the preceding period of nerve stimulation.

4. The method of claim 1, wherein the interval period with no nerve stimulation has a duration of at least approximately 5 times the duration of the preceding period of nerve stimulation.

5. The method of claim 1, wherein the interval period with no nerve stimulation has a duration of at least approximately 10 times the duration of the preceding period of nerve stimulation.

6. The method of claim 1, wherein a form of energy selected from electrical energy, light, vibration, and ultrasonic vibration is applied to stimulate the sympathetic nerve.

7. The method of claim 1, further comprising:
placing an electrode in the vicinity of a portal vein or hepatic artery of the patient;
connecting an electronic module to the electrode;
implanting the electronic module into the patient's body; and
applying an electrical current through the electrode to stimulate a perivascular sympathetic nerve.

8. The method of claim 7, further comprising:
implanting the electrode within the patient using a percutaneous transhepatic approach.

9. The method of claim 7, further comprising:
implanting the electrode within the patient using a intravenous intrahepatic approach.

10. The method of claim 7, further comprising:
implanting the electrode within the patient using an arterial catheterization approach.

11. The method of claim 7, further comprising:
implanting the electrode within the patient using a laparoscopic approach.

12. The method of claim 7, further comprising:
implanting the electrode within the patient using an endoscopic approach.

13. The method of claim 1, further comprising:
placing an electrode in a lumen of a portal vein or hepatic artery of the patient; and
applying an electrical current through the electrode to stimulate a perivascular sympathetic nerve;
wherein the electrode does not obstruct blood flow through the lumen of the portal vein or hepatic artery of the patient.

14. The method of claim 1, further comprising:
placing a leadless electrode in the vicinity of a portal vein or hepatic artery of the patient, wherein the leadless electrode is integrated with an electronic module; and
applying an electrical current from the electronic module through the electrode to stimulate a perivascular sympathetic nerve.

15. The method of claim 8, further comprising:
programming or controlling the leadless electrode wirelessly using an electronic module positioned external to the patient's body.

16. The method of claim 1, further comprising:
verifying an increase in renal function, and permanently ablating at least one perivascular sympathetic nerve.

17. A method of increasing renal function in a patient, comprising:
periodically stimulating a sympathetic nerve located in a perivascular area in the vicinity of a portal vein or hepatic artery of the patient, each period of nerve stimulation being followed by an interval period with no nerve stimulation in which glomerular filtration rate of the patient is increased, wherein the interval period with no nerve stimulation has a duration of at least approximately 2.25 times a duration of the preceding period of nerve stimulation.

18. The method of claim 17, wherein the interval period with no nerve stimulation has a duration of approximately 10 minutes and the period of nerve stimulation has a duration of approximately 2 minutes.

19. The method of claim 17, wherein the interval period with no nerve stimulation has a duration of approximately 10 minutes and the period of nerve stimulation has a duration of approximately 1 minute.

20. A method of treating for contrast-induced nephropathy in a patient, comprising:
injecting a radiopaque contrast medium into the patient's vascular system; and
periodically stimulating a sympathetic nerve located in a perivascular area in the vicinity of a portal vein or hepatic artery of the patient, each period of nerve stimulation being followed by an interval period with no nerve stimulation during which glomerular filtration rate of the patient is increased to clear the radiopaque contrast medium from the patient's body.
